(19) United States
(12) Patent Application Publication
(10) Pub. No.: US 2026/0232196 A1
(43) Pub. Date: Aug. 13, 2026

(54) DEVICES, METHODS, AND SYSTEMS FOR MAKING OPHTHALMIC MEASUREMENTS WITH THE AURICLE AS A REFERENCE

(71) Applicants: Soheab Ugradar, Los Angeles, CA (US); Raymond Douglas, Las Vegas, NV (US)

(72) Inventors: Soheab Ugradar, Los Angeles, CA (US); Raymond Douglas, Las Vegas, NV (US)

(21) Appl. No.: 19/050,943

(22) Filed: Feb. 11, 2025

(51) Int. Cl. A61B 5/00; G06T 7/00

(52) U.S. Cl. CPC A61B 5/0059; A61B 5/7264; A61B 5/742; G06T 7/0012; G06T 2207/10004; G06T 2207/30041

(57) ABSTRACT

A method of ophthalmic measuring comprising: providing a digital image, the digital image comprises a depiction of an auricle and an oculus, positioned on a same side of a face; converting the digital image, into a digital representation of the auricle and into a digital representation of the oculus; defining a plurality of boundaries of the oculus by identifying distinguishing features to enable recognition of a corneal apex of the oculus; defining a plurality of boundaries of the auricle by identifying distinguishing auricle features to enable recognition of anatomical auricle reference points; mapping the auricle by identifying the anatomical auricle reference points that were recognized; creating measurement vectors, wherein each of the measurement vectors originate from the anatomical auricle reference points and extend to the corneal apex; and combining the measurement vectors, such that a resultant vector is created/mathematically determined; wherein the resultant vector represents a proptosis measurement.

### FIELD OF USE

The present disclosure relates, in general, to devices, methods, and systems for making ophthalmic measurements with the auricle as a reference. More specifically, the present disclosure relates to ophthalmic measurements for purposes of making a medical diagnosis related to proptosis.

### BACKGROUND

Generally, ophthalmic measurements are taken using the temporal orbital rim for each oculus (“eye”) or eyelid and the apex of the cornea.

Using exophthalmometers to make ophthalmic measurements is difficult with patients having severe upper eyelid swelling, ptosis, hyper-deviated eyes, and poor cooperation.

Using computed topography (“CT”) assessment of proptosis on cross-sectional imaging is difficult and dependent on the study being acquired in the correct plane: the plane of the study must be parallel to the head of the optic nerve and the lens, and the patient must have their eyes open and be looking forward with no eye movement.

Magnetic resonance imaging (“MRI”) may also be used in evaluation due to its multiplanar and inherent contrast capabilities. Use of MRI prevents ionizing radiation of the orbits and risk of radiation-induced cataracts. The imaging findings are similar to those described above for CT.

Because each of these studies is dependent on many external conditions to measurement, what is needed is a method of making ophthalmic measurements using a reference point that is not dependent related to the ophthalmic area of concern.

### SUMMARY

To minimize the limitations in the prior art, and to minimize other limitations that will become apparent upon reading and understanding the present specification, the present disclosure discloses new and useful devices, methods, and systems of making ophthalmic measurements.

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The problem to be solved is making consistent and reliable ophthalmic measurements using devices, systems, and methods that are free from external error influences.

The object of this disclosure is to disclose devices, methods, and systems for making ophthalmic measurements by forming an anatomical reference point based on the auricle of a subject.

The devices, methods, and systems of the present disclosure teach creating an image or video stream of an oculus and an auricle, determining measurements between the two, analyzing the measurements, and providing diagnostic information related to proptosis, gaze, eye color, eye movement, eye disease inflammation, and height relative to the corneal apex.

The present disclosure provides for obtaining a measurement of proptosis related to multiple points on the ear that form at least one digital and anatomical reference point that is used to align the patient so that the anterior projection of the eye (how far forward the eye is) can be consistently measured from a fixed region of the head.

One embodiment of the present disclosure may be a smart phone app that is in communication with (possibly via the internet) a software program that uses photographs or video taken by the camera of the smart phone to analyze (which includes machine learning and other advanced algorithms) eyelid and brow height, eye color, proptosis, and degree of eye movement. Eye color may be analyzed using the L*a*b* color technique. The CIELAB color space, sometimes referred to as L*a*b*, is a color space defined by the International Commission on Illumination (abbreviated CIE), which expresses color as three values: L* for perceptual lightness and a* and b* for the four unique colors of human vision: red, green, blue, and yellow.

Another embodiment may be a specific purpose device built specifically for making and processing ophthalmic measurements.

One embodiment may be a method of ophthalmic measuring, the method comprising: by an ophthalmic measurement device: providing a digital image, the digital image comprises a depiction of an auricle and an oculus; the auricle and the oculus are preferably positioned on a same side of a face; converting the digital image, such that the depiction of the auricle and the oculus may be rendered into a digital representation of the auricle and into a digital representation of the oculus; defining boundaries of the digital representation of the oculus by identifying distinguishing oculus features to enable recognition of a corneal apex of the oculus; defining boundaries of the digital representation of the auricle by identifying distinguishing auricle features to enable recognition of anatomical auricle reference points; mapping the digital representation of the auricle by identifying the anatomical auricle reference points that were recognized; creating measurement vectors, each of the measurement vectors originate from the anatomical auricle reference points and extend to the corneal apex; and combining the measurement vectors, such that a resultant vector may be created/mathematically determined; the resultant vector represents a proptosis measurement. Determining, based on the proptosis measurement, one of a mid-proptosis, a mild proptosis, and a severe proptosis. The digital image may be captured with a digital camera. Scanning the photograph, such that the digital image may be created. The distinguishing auricle features may comprise a lobule, a helix, and a tragus. The distinguishing oculus features may comprise a pupil, a canal of Schlemm, an iris, and a cornea. The anatomical auricle points may comprise: a triangular fossa; a superior crus of antihelix; a helix; a scapha; an antihelix stem; a cymba, a cavum, an antitragus; a lobe; an incisura; a tragus; a crus helix; and an inferior crus of antihelix. The measurement vectors may be a minimum of three measurement vectors.

Another embodiment may be a non-transitory computer readable medium with computer executable instructions for ophthalmic measuring, the computer readable medium having computer executable instructions comprising: providing an electronic application; providing a digital image; receiving, by the electronic application, the digital image; the digital image may comprise a depiction of an auricle and an oculus; the auricle and the oculus are preferably positioned on a same side of a face; converting, by the electronic application, the digital image, such that the depiction of the auricle and the oculus are rendered into a digital representation of the auricle and into a digital representation of the oculus; processing, by the electronic application, the digital representation of the auricle, such that the electronic application identifies anatomical auricle reference points; processing, by the electronic application, the digital representation of the oculus, such that the electronic application identifies a corneal apex of the digital representation of the oculus; creating, by the electronic application, measurement vectors, each of which originates from one of the anatomical auricle reference points and terminates at the corneal apex; combining, by the electronic application, the measurement vectors, such that a resultant vector, may be created/mathematically determined; the resultant vector represents a proptosis measurement. Determining, by the electronic application, a diagnosis of a mid-proptosis, a mild proptosis, and a severe proptosis, based on the resultant vector. Capturing the digital image with a digital camera. Scanning the photograph, such that the digital image may be created. Identify the anatomical auricle reference points comprises: defining, by the electronic application, boundaries of the digital representation of the auricle by identifying, by the electronic application, distinguishing auricle features to enable recognition of the anatomical auricle reference points; and mapping, by the electronic application, the digital representation of the auricle by identifying the anatomical auricle reference points that were recognized.

Another embodiment may be a system for obtaining ophthalmic measurements for the purpose of diagnosing proptosis, comprising: an ophthalmic measurement device, which may comprise a digital camera, a display, an electronic data processing unit, and a memory; an electronic application, at least a portion of which may be stored in the memory and processed by the electronic data processing unit; the digital camera may capture a digital image that may comprise a depiction of an auricle and of an oculus that are preferably on a same side of a face; the electronic application, running on the ophthalmic measurement device: renders, respectively, the depiction of the auricle and the oculus into a digital representation of the auricle and into a digital representation of the oculus; defines boundaries of the digital representation of the oculus by identifying distinguishing oculus features to enable recognition of a corneal apex of the oculus; defines boundaries of the digital representation of the auricle by identifying distinguishing auricle features to enable recognition of anatomical auricle reference points; digitally maps the digital representation of the auricle by identifying the anatomical auricle reference points that were defined; creates measurement vectors, each of the measurement vectors originates from one or more of the anatomical auricle reference points and extends to the corneal apex; and combines the measurement vectors, such that a resultant vector may be created; wherein the resultant vector represents a proptosis measurement. Further comprising an artificial intelligence engine. The electronic application running on the ophthalmic measurement device determines, based on the proptosis measurement, one of a mid-proptosis, a mild proptosis, and a severe proptosis. The distinguishing auricle features may comprise a lobule, a helix, and a tragus. The distinguishing oculus features may comprise a pupil, a canal of Schlemm, an iris, and a cornea. The anatomical auricle points may comprise: a triangular fossa; a superior crus of antihelix; a helix; a scapha; an antihelix stem; a cymba, a cavum, an antitragus; a lobe; an incisura; a tragus; a crus helix; and an inferior crus of antihelix. The measurement vectors may be a minimum of three measurement vectors.

It is an object to overcome the limitations of the prior art.

These, as well as other components, steps, features, objects, benefits, and advantages, will now become clear from a review of the following detailed description of illustrative embodiments, the accompanying drawings, and the claims.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an illustration of the anatomy of an oculus.

FIG. 2 is an illustration of the anatomy of an auricle.

FIG. 3 is an illustration of mapped points and measurement vectors to a corneal apex reference point.

FIG. 4 is an illustration of the multiple measurement vector creation of a resultant vector.

FIG. 5 is a flow block diagram of one embodiment of a method of determining proptosis.

FIG. 6 is a flow block diagram of one embodiment of an automated application determining proptosis.

FIG. 7 is a flow block diagram of one embodiment of an automated application determining gaze.

FIG. 8 is a flow block diagram of one embodiment of an automated application quantifying thyroid eye disease inflammation.

FIG. 9 is a flow block diagram of one embodiment of an automated application measuring a height above the corneal apex.

FIG. 10 is an illustration of one embodiment of an ophthalmic measurement device.

FIG. 11 is a system block diagram of one embodiment of an ophthalmic measurement system for diagnosing proptosis.

### DETAILED DESCRIPTION OF THE ILLUSTRATIVE EMBODIMENTS

In the following detailed description of various embodiments of the present disclosure, numerous specific details are set forth in order to provide a thorough understanding of various aspects of one or more embodiments of the present disclosure. However, one or more embodiments of the present disclosure may be practiced without some or all of these specific details. In other instances, well-known methods, procedures, and/or components have not been described in detail so as not to unnecessarily obscure aspects of embodiments of the present disclosure.

While multiple embodiments are disclosed, still other embodiments of the devices, systems, and methods of the present disclosure will become apparent to those skilled in the art from the following detailed description, which shows and describes illustrative embodiments of the devices, systems, and methods of the present disclosure. As will be realized, the devices, systems, and methods of the present disclosure are capable of modifications in various obvious aspects, all without departing from the spirit and scope of the present disclosure. Accordingly, the screenshot figures, and the detailed descriptions thereof, are to be regarded as illustrative in nature and not restrictive. Also, the reference or non-reference to a particular embodiment of the devices, systems, and methods of the present disclosure shall not be interpreted to limit the scope of the present disclosure.

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers, or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-40% from the indicated number or range of numbers.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

Furthermore, the one or more versions may be implemented as a method, apparatus, or article of manufacture using standard programming and/or engineering techniques to produce entirely hardware embodiment, an entirely software embodiment, or an embodiment combining software and hardware embodiments. Furthermore, the systems and methods may take the form of non-transitory computer readable media. More particularly, the present methods and systems may take the form of web-implemented computer software or a computer program product. Any suitable computer-readable storage medium may be utilized including, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick).

Those skilled in the art will recognize many modifications may be made to this configuration without departing from the scope of the disclosed embodiments.

Embodiments of the systems and methods are described below with reference to schematic diagrams, block diagrams, and flowchart illustrations of methods, systems, apparatuses, and computer program products. It will be understood that each block of the block diagrams, schematic diagrams, and flowchart illustrations, and combinations of blocks in the block diagrams, schematic diagrams, and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

In the following description, certain terminology is used to describe certain features of the various embodiments of the device, method, and/or system. For example, as used herein, the terms “computer” and “computer system” generally refer to any device that processes information with an integrated circuit chip and/or central processing unit (CPU).

As used herein, the terms “software” and “application” refer to any set of machine-readable instructions on a machine, web interface, and/or computer system” that directs a computer's processor to perform specific steps, processes, or operations disclosed herein.

As used herein, the term “computer-readable medium” refers to any storage medium adapted to store data and/or instructions that are executable by a processor of a computer system. The computer-readable storage medium may be a computer-readable non-transitory storage medium and/or any non-transitory data storage circuitry (e.g., buggers, cache, and queues) within transceivers of transitory signals. The computer-readable storage medium may also be any tangible computer readable medium. In various embodiments, a computer readable storage medium may also be able to store data, which is able to be accessed by the processor of the computer system.

As used herein, the term “color of sclera” refers to any non-white color of the sclera (the “white” part of the eye) used to assist in a health and disease diagnosis.

As used herein the term “oculus” refers to the anatomical eye of an animal.

As used herein, the term “proptosis” refers to the forward protrusion of an eyeball from its natural position.

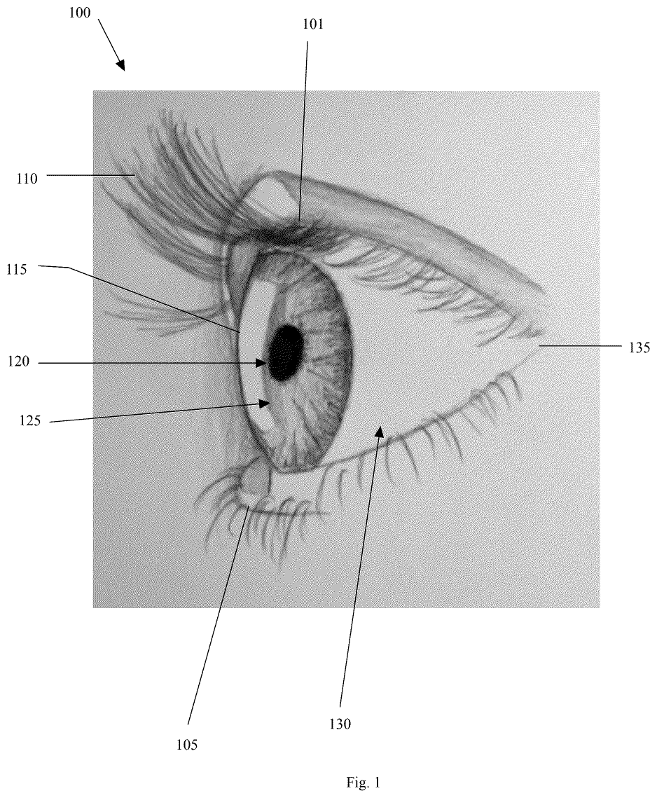

FIG. 1 is an illustration of the anatomy of an oculus. As shown, oculus 100 may have upper eyelid 101, corner of oculus 135, sclera 130, lower eyelid 105, iris 125, pupil 120, cornea 115, and eye lashes 101.

The natural shape of cornea 115 is prolate or steeper centrally than peripherally. The natural prolate corneal shape results in an aspheric optical system, which reduces spherical aberration and therefore minimizes fluctuations in refractive error as the pupil changes size.

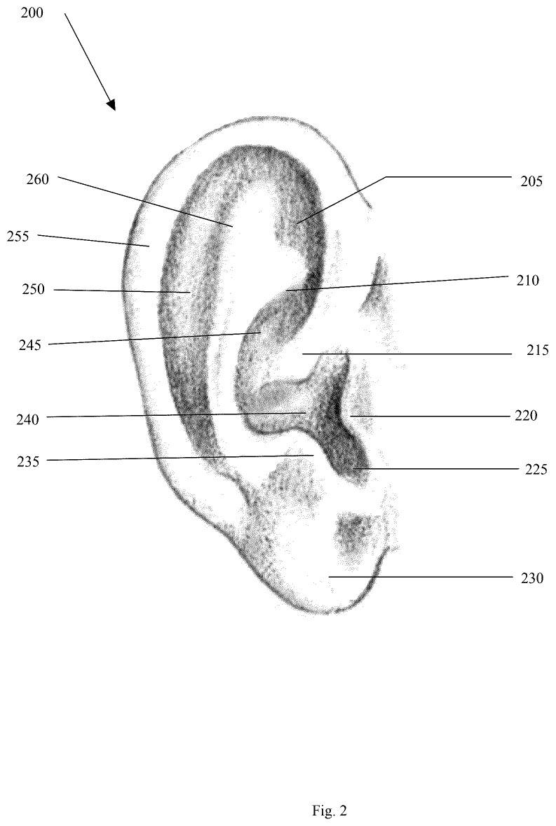

FIG. 2 is an illustration of the anatomy of an auricle. As shown, the ear, or auricle 200 may comprise various features, including, but not limited to, triangular fossa 205, inferior crus of antihelix 210, crus helix 215, tragus 220, incisura 225, lobe 230, antitragus 235, cavum 240, cymba 245, scapha 250, helix 255, and superior crus of antihelix 260.

Auricle 200 is the visible part of the ear outside the head. It is the elastic cartilaginous part of the external ear that projects outwards from the side of the head and is covered by skin.

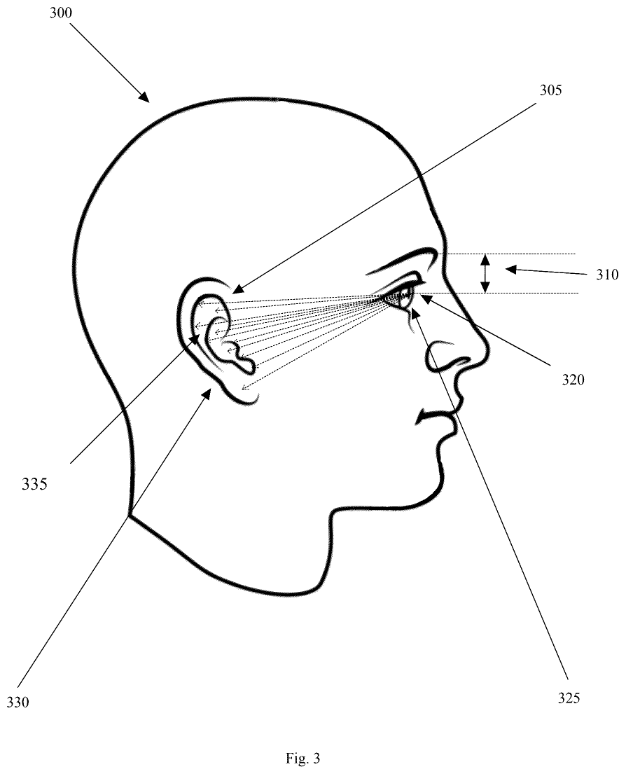

FIG. 3 is an illustration of mapped points and measurement vectors to a corneal apex reference point. Map 300 shows a plurality of measurement vectors 305 (between corneal apex 325 and auricle 330), height above 310 (between corneal apex 320 and, for example, eyebrow), corneal apex 320, oculus 320, auricle 330, and a plurality of anatomical auricle reference points 335. Although FIG. 3 necessarily shows map 300 with analog facial features, these features are preferably represented digitally (electronically as memory or processing data).

Measurement vectors 305 preferably originate from the corneal apex 320 and extend to a plurality of anatomical auricle reference points 335. Although only eleven measurement vectors 305 are shown on map 300, an essentially infinite number of additional measurement vectors 305, may be utilized. Any increase in the number of additional measurement vectors 305 to anatomical auricle reference points 335 may improve the repeatability and accuracy of any measurements. Increasing the number of measurement vectors 305 may also reduce the size of anatomical auricle reference points 335, which may reduce integration errors in measurements.

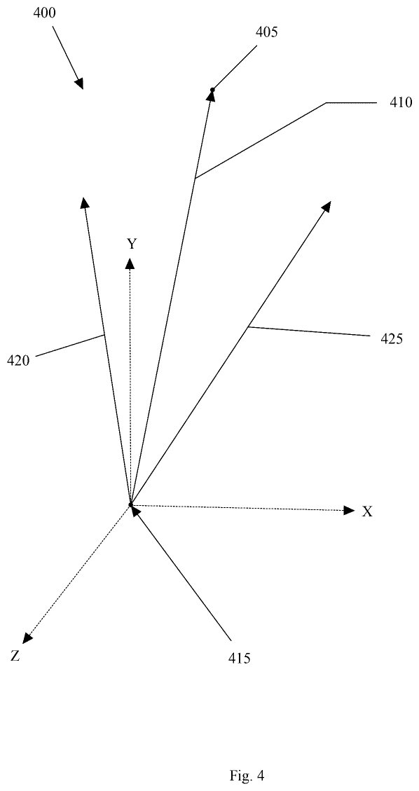

FIG. 4 is an illustration of the multiple measurement vector creation of a resultant vector. Resultant vector creation 400 may be represented in a three-dimensional cartesian coordinate system having an X, Y, and Z plane. Resultant vector creation 400 may comprise a first measurement vector 420, Nth number of measurement vectors 425, resultant vector 410, resultant first endpoint 415, and resultant second endpoint 405. Resultant vector 410 may be created from a combination of first measurement vector 420 and Nth measurement vectors 425. Although only two measurement vectors are shown in FIG. 4, it is well known in the art that any number of measurement vectors in combination may represent resultant vector 410. It is preferable that a plurality of measurement vectors be used to create resultant vector 410.

The resultant vector 410 may include a first endpoint of 415 and a second endpoint of 405 The magnitude of resultant vector 410 may represent a physical distance between an anatomical reference point and a corneal apex. An increase in the number of Nth measurement vectors 425 may increase the reliability and accuracy of a measurement interpreted by resultant vector 410.

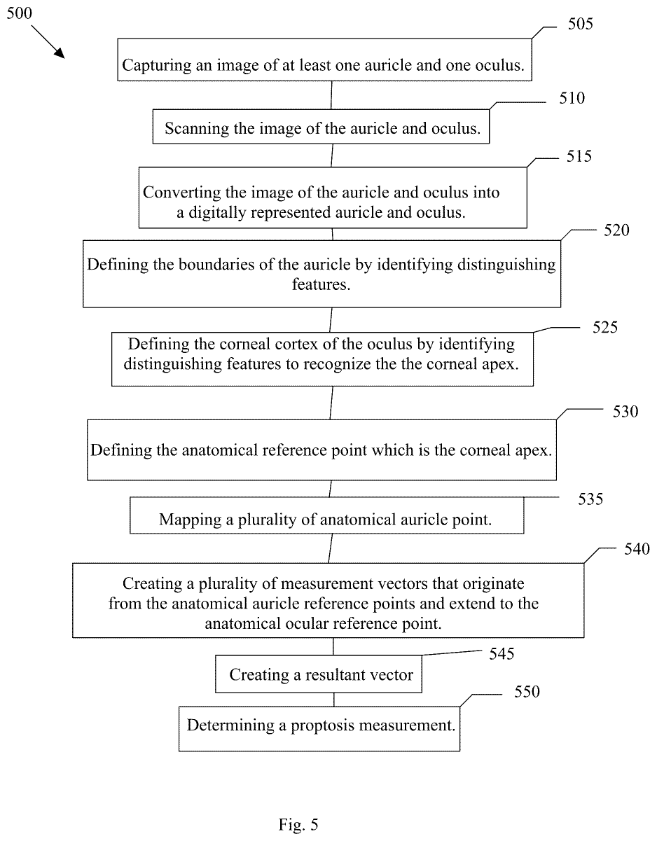

FIG. 5 is a flow block diagram of one embodiment of a method of determining proptosis. The method of ophthalmic measuring 500 may comprise capturing 505 an image (or a stream of images) of an auricle and an oculus that are on the same side of a face; scanning 510 the image of the auricle and oculus, scanning may include, but is not limited to, accepting a digital image into memory or optically scanning a photograph (digitization); converting 515 the scanned image of the auricle and the oculus into a digitally represented auricle and oculus, the digitally represented auricle and oculus may be reduced to data held in memory, and alternatively represented on a graphical display, The process of converting a photo to a digital picture is called digitization, digitization involves scanning the photo, which breaks it down into pixels that contain color and intensity information, the scanned image is then stored on a disk or memory card, and output devices like printers and monitors convert the digital information back into pictures; defining 520 the boundaries of the auricle by identifying distinguishing features of, or that represent, the auricle, e.g., as shown in FIG. 2 including, but not limited to, triangular fossa 205, inferior crus of antihelix 210, crus helix 215, tragus 220, incisura 225, lobe 230, antitragus 235, cavum 240, cymba 245, scapha 250, helix 255, and superior crus of antihelix 260; defining 525 or determining a corneal apex of the oculus, by using the identified distinguishing features to recognize the corneal apex; defining 530 an anatomical ocular reference point, such as the corneal apex; mapping 535 the auricle by identifying a number of anatomical auricle points, it is preferable to use a large number of anatomical auricle points as reliability and accuracy of the succeeding measurements increases with an increase in the number of anatomical points; creating 540 measurement vectors, the measurement vectors may originate from the anatomical auricle points and extend to the anatomical ocular reference point, but the vectors may alternatively originate from the anatomical ocular reference point and extend to the plurality of anatomical auricle points; creating 545 or determining a resultant vector from the several measurement vectors, the resultant vector may represent a proptosis measurement; and determining 550 or diagnosing proptosis based on the measurement as mid-proptosis, mild proptosis, or severe proptosis.

It is preferable that the corneal apex be the cornea's point of maximum curvature.

Mid-proptosis is in the range of 21 to 23 millimeters.

Mild proptosis is in the range 24-27 millimeters.

Severe proptosis is greater than 28 millimeters.

It is well known that the front of an oculus, a white-to-white measurement, is a standard 11.64 millimeters (mm) for females and 11.77 mm for males. Using these two standards, a pixel-to-mm ratio may be used to provide a scale/measurement for measurements made related to any profile video or photo that includes an ear and an eye.

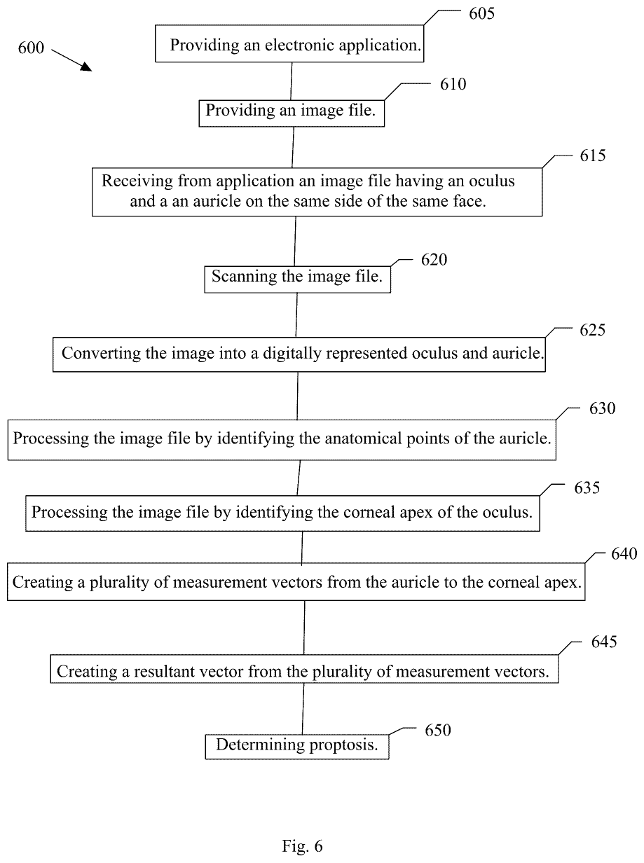

FIG. 6 is a flow block diagram of one embodiment of an automated application determining proptosis. The method of ophthalmic measuring 600 may comprise the steps: providing 605 an electronic software application, the electronic application may include an interface that includes an artificial intelligence framework; providing 610 a digital image (or digital image file), that may be from a digital camera or a physical photograph (which is then digitally scanned); receiving 615 by the electronic application the image, which may be stored in the memory of a local or remote device, the image preferably comprises a depiction of an auricle and an oculus from the same side of a face; scanning 620 by the electronic application (which is preferably a software program), the digital image of the auricle and the oculus; converting 625 the image of the ear and eye by the electronic software application to a digital representation of the ear and the eye, the digital representation may be data then stored in memory or a digital three dimensional graphical image with anatomical mapping points; processing 630, by the electronic software application, the digital image and identifying a plurality of anatomical points of the auricle; processing 635, by the electronic software application, the digital image and identifying a corneal apex of the oculus, the corneal apex is preferable the cornea's point of maximum curvature; creating 640, by the electronic software application, measurement vectors that may originate from the anatomical auricle points and culminate at the corneal apex; creating 645, by the electronic software application, a resultant vector, the resultant vector may be a resultant vector of the measurement vectors; and determining 650, by the electronic software application, a mid-proptosis, a mild proptosis, or a severe proptosis diagnosis based on the resultant vector measurement.

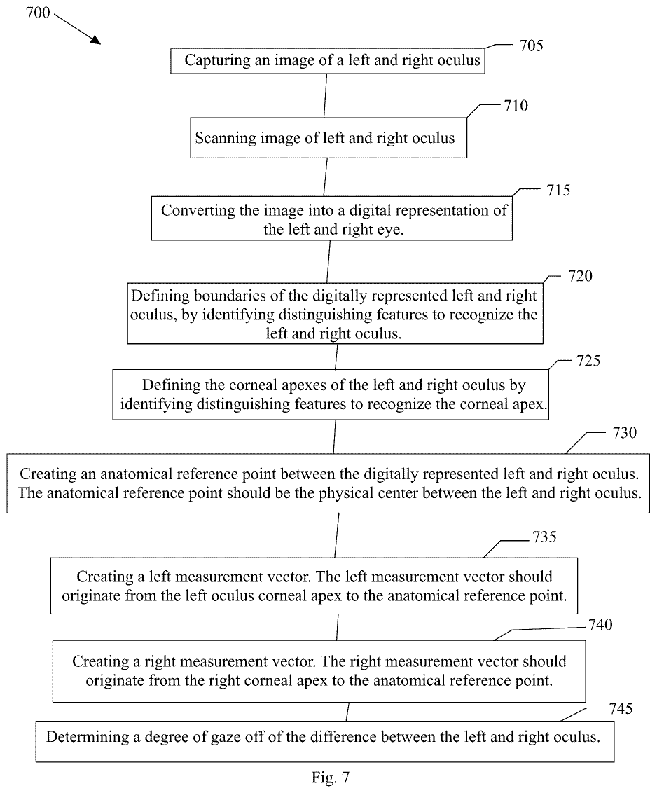

FIG. 7 is a flow block diagram of an automated application determining gaze. A method of measuring front eye deviation 700, the method may include capturing 705 an image of a left and right oculus; scanning 710 the image of the left and the right oculus, scanning may include direct digital transfer of a digital picture or digitally scanning a physical photograph; converting 715 the image of the of left oculus and the right oculus into a digitally represented left oculus and a digitally represented right oculus, the digital representations may be data in memory or may include a multi-dimensional digital graphical representation of the two eyes; defining 720 boundaries of the digitally represented left oculus and the digitally represented right oculus, by identifying distinguishing features of the digitally represented left oculus and the digitally represented right oculus, and using the distinguishing features to recognize and define the boundaries; defining 725 the corneal apexes of the digitally represented left oculus and the digitally represented right oculus, by identifying distinguishing features of the digitally represented left oculus corneal apex and the digitally represented right oculus corneal apex, and using those distinguishing features to recognize the apexes, it is preferable that each corneal apex is the point maximum curvature; creating 730 a digital anatomical reference point between the digitally represented left oculus and the digitally represented right oculus, the digital anatomical reference point may preferably be at the center or center point between the left eye and the right eye; creating 735 a left measurement vector, the left measurement vector preferably originates from the digitally represented left oculus corneal apex and extends to the digital anatomical reference point; creating 740 a right measurement vector, wherein the right measurement vector originates from the digitally represented right oculus corneal apex to the digital anatomical reference point; and determining 745 degree of gaze, based on the difference between the left measurement vector and the right measurement vector. It is preferable that the image be a front elevation view showing a patient.

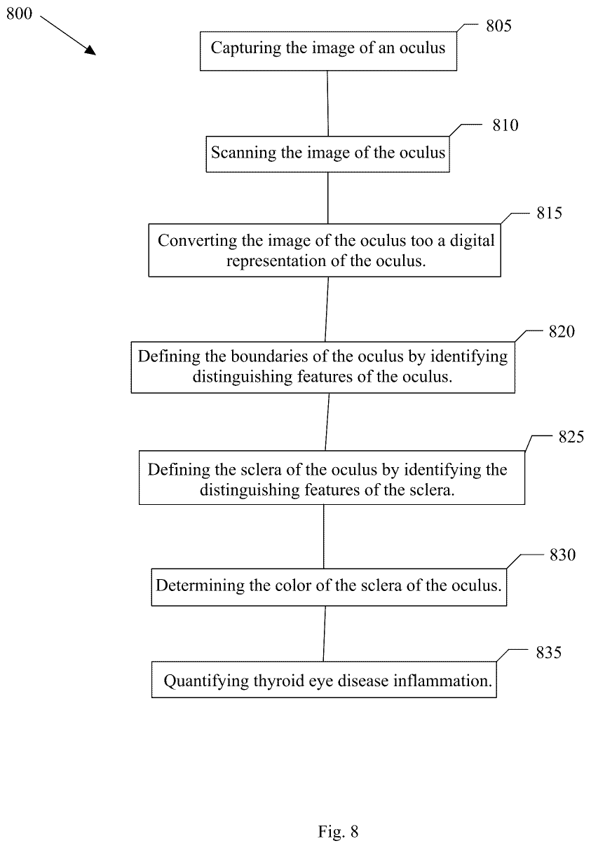

FIG. 8 is a flow block diagram of one embodiment of an automated application quantifying thyroid eye disease inflammation. The method of determining inflammation 800 may comprise: capturing 805 an image of at least one oculus; scanning 810 or digitizing the image of the oculus, and the scanning may include digitally transferring an image or physically scanning a photograph; converting 815 the image of the oculus into a digitally represented oculus, the digital representation of the oculus may include a multi-dimensional graphical display; defining 820 boundaries of the digitally represented oculus, by identifying distinguishing features that then enable recognition the digitally represented oculus, including boundaries; defining 825 the sclera of the digitally represented oculus, by using the identified distinguishing; determining 830 the color of the sclera of the digitally represented oculus, preferably using L*a*b* color; quantifying 835, based on the color of the sclera, inflammation of the oculus in the image. Inflammation may be caused by and/or be a symptom of thyroid eye disease.

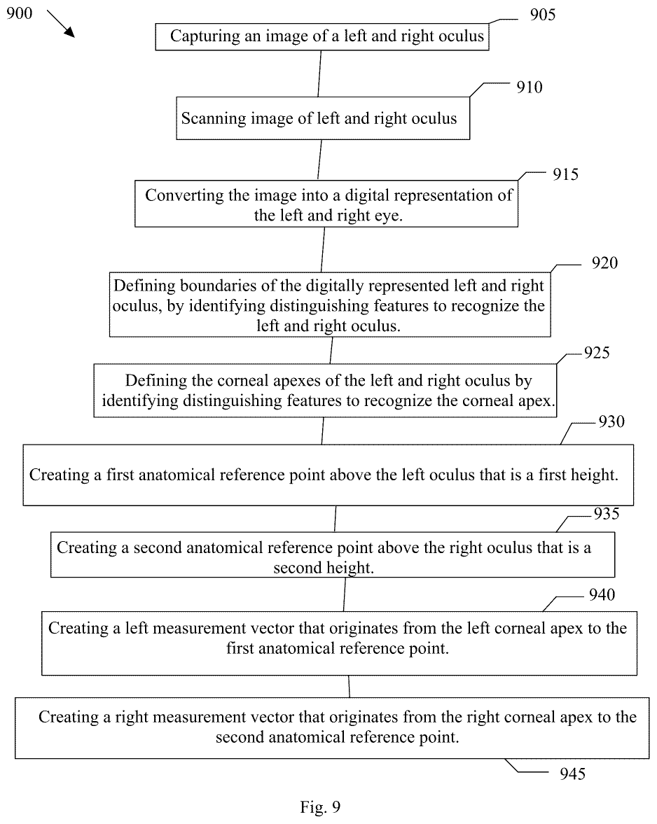

FIG. 9 is a flow block diagram of one embodiment of an automated application for measuring a height above the corneal apex. The method of measuring height to the pupil position 900 may comprise: capturing 905 an image of a left and right oculus; scanning 910 the image of the left and the right oculus, scanning may include digitally importing the image or physically scanning a photograph; converting 915 the image of the left oculus and right oculus into a digitally represented left oculus and a digitally represented right oculus, the digital representations may include a multi-dimensional graphical display; defining 920 boundaries of the digitally represented left oculus and the digitally represented right oculus, by identifying distinguishing features that then enable recognition of the digitally represented left oculus and the digitally represented right oculus; defining 925 a corneal apex of the digitally represented left oculus and the digitally represented right oculus, by using the identified distinguishing features; creating 930 a first anatomical reference point above the digitally represented left oculus, the first anatomical reference point may be a first height and is preferably a digital reference point; creating 935 a second anatomical reference point above the digitally represented right oculus, the second anatomical reference point may be a second height and is preferably a digital reference point; creating 940 a left measurement vector, the left measurement vector may originate from the digitally represented left oculus corneal apex to the first anatomical reference point, or may originate from the first anatomical reference point to the digitally represented left oculus corneal apex; and creating 945 a right measurement vector, the right measurement vector may originate from the digitally represented right oculus corneal apex to the second anatomical reference point, or the second anatomical reference point to the digitally represented right oculus corneal apex. The first height and second height may define an eye-brow height. Alternatively, the first height and second height define an eyelid height.

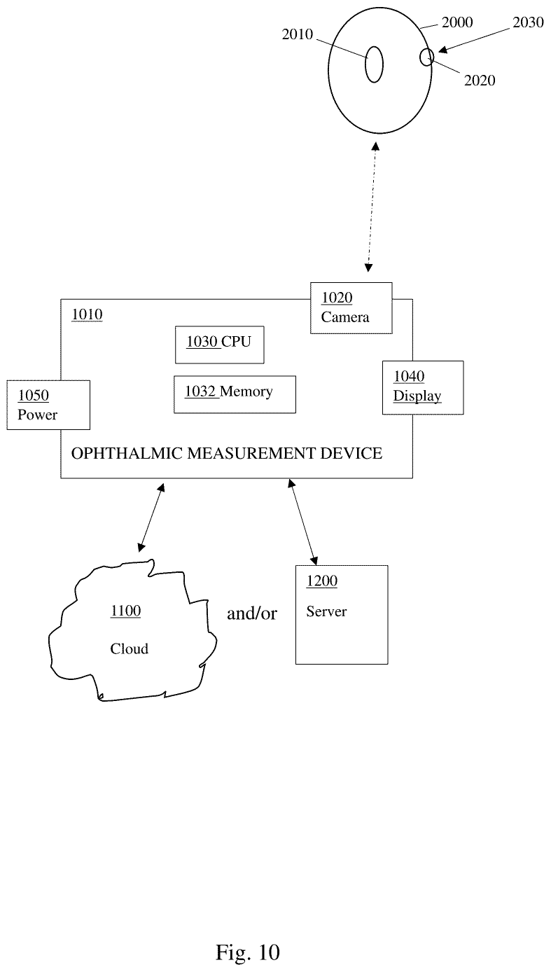

FIG. 10 is an illustration of one embodiment of an ophthalmic measurement device. As shown in FIG. 10, ophthalmic measurement device 1010 may comprise camera 1020, display 1040, electronic data processing unit 1030, memory 1032, and power source 1050. Ophthalmic measurement device 1010 may be configured to run various software applications, such as an App, that allow the ophthalmic measurement device 1010 to take images of a face 2000, process and/or digitize the images, create digital representations of various facial features, such as eye 2020 and ear 2010, define and identify distinguishing characteristics of the digitized images, such that reference points and feature points may be recognized, such as corneal apex 2030, map certain anatomical points on a 3D digital rendering of ear 2010, create a plurality of measurement vectors between two or more of the recognized reference points, and create a resultant vector. That same program or a related program may then provide a diagnosis, such as proptosis, from the resultant vector. Ophthalmic measurement device 1010 may be a smartphone, where the camera 1020, power supply 1050, and display 1040 are integrated into ophthalmic measurement device 1010. In this embodiment, power supply 1050 may be a battery, preferably a rechargeable battery. In other embodiments, ophthalmic measurement device 1010 may be a separate unit that is in wired or wireless connectivity to camera 1020, display 1040, and power supply 1050. In this embodiment, power supply 1050 may be an outlet connection to an A/C socket. In other embodiments, ophthalmic measurement device 1010 may be connected, wired or wirelessly, to the Cloud 1100, or one or more servers 1200, which may provide the processing software for the processing, digitizing, identifying, recognizing, rendering, measuring, and creating, as detailed above.

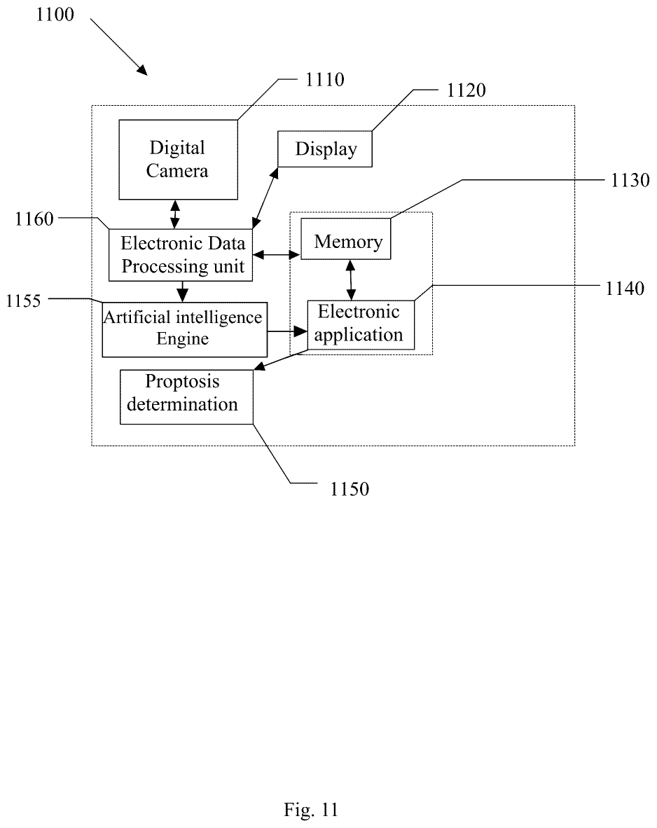

FIG. 11 is a system block diagram of one embodiment of an ophthalmic measurement system for diagnosing proptosis. The ophthalmic measurement system 1100 of the present disclosure is generally configured to measure proptosis that may comprise a digital camera 1110, a display 1120, a memory 1130, an electronic application 1140, and a proptosis determination 1150, an artificial intelligence engine 1155, and an electronic data processing unit 1160.

The digital camera 1110 may be but is not limited to an optical image-capturing device such as a digital single-lens reflex camera, a computer video camera, a telephone camera, or the like capable of capturing an image and converting the image to digital data.

Display 1120 may be, but not limited to, a standard computer display, a camera display, or a telephone display. In one embodiment, display 1120 may allow for the manual creation of measurement vectors.

Memory 1130 may store digital data to be processed by an electronic data processing unit.

Electronic application 1140 may include a non-transitory computer-readable medium with computer-executable instructions for ophthalmic measuring. The computer-readable medium may include computer-executable instructions.

Proptosis determination 1150 may include a mid-proptosis, a mild proptosis, and a severe proptosis.

Artificial intelligence engine 1155 may be useful.

Electronic data processing unit 1160 may include a standard computer processor but is not limited to it. In an alternate embodiment, it may be a specifically designed data processing unit.

One embodiment may be a system 1100 for obtaining ophthalmic measurements for the purpose of diagnosing proptosis, comprising: an ophthalmic measurement device, such as device 1010, which may comprises digital camera 1110, display 1120, electronic data processing unit 1160, and memory 1130; electronic application 1140, at least a portion of which is stored in memory 1130 and processed by electronic data processing unit 1160. Digital camera 1110 may capture a digital image that may comprise a depiction of an auricle and of an oculus that are on a same side of a face. Electronic application 1140 may (1) render, respectively, the depiction of the auricle and the oculus into a digital representation of the auricle and into a digital representation of the oculus; (2) define a plurality of boundaries of the digital representation of the oculus by identifying one or more distinguishing oculus features to enable recognition of a corneal apex of the oculus; (3) define a plurality of boundaries of the digital representation of the auricle by identifying one or more distinguishing auricle features to enable recognition of a plurality of anatomical auricle reference points; (4) digitally map the digital representation of the auricle by identifying the plurality of anatomical auricle reference points that were defined; (4) create a plurality of a measurement vectors, wherein each of the plurality of measurement vectors originates from one or more of the plurality of anatomical auricle reference points and extends to the corneal apex; and (5) combines the plurality of measurement vectors, such that a resultant vector is created. Preferably, the resultant vector represents a proptosis measurement. In another embodiment, the system 1100 may further comprise an artificial intelligence engine 1155; wherein artificial intelligence engine 1155 is useful.

Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

Some portions of the preceding detailed descriptions have been presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it should be appreciated that throughout the present disclosure, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display devices.

The processes or methods depicted in the figures may be performed by processing logic that comprises hardware (e.g., circuitry, dedicated logic, etc.), firmware, software (e.g., embodied on a non-transitory computer readable medium), or a combination thereof. Although the processes or methods are described above in terms of some sequential operations, it should be appreciated that some of the operations described may be performed in a different order. Moreover, some operations may be performed in parallel rather than sequentially.

In addition, the various illustrative logical blocks, modules, and circuits described in connection with certain embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize many modifications may be made to this configuration without departing from the scope of the disclosed embodiments.

The foregoing description of the preferred embodiment has been presented for the purposes of illustration and description. While multiple embodiments are disclosed, still other embodiments will become apparent to those skilled in the art from the above detailed description. These embodiments are capable of modifications in various obvious aspects, all without departing from the spirit and scope of protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

What is claimed is:

1. A method of ophthalmic measuring, the method comprising:
by an ophthalmic measurement device:
providing a digital image, wherein said digital image comprises a depiction of an auricle and an oculus;
wherein said auricle and said oculus are positioned on a same side of a face;
converting said digital image, such that said depiction of said auricle and said oculus are rendered into a digital representation of said auricle and into a digital representation of said oculus;
defining a plurality of boundaries of said digital representation of said oculus by identifying one or more distinguishing oculus features to enable recognition of a corneal apex of said oculus;
defining a plurality of boundaries of said digital representation of said auricle by identifying one or more distinguishing auricle features to enable recognition of a plurality of anatomical auricle reference points;
mapping said digital representation of said auricle by identifying said plurality of anatomical auricle reference points that were recognized;
creating a plurality of a measurement vectors, wherein each of said plurality of measurement vectors originate from one or more of said plurality of anatomical auricle reference points and extend to said corneal apex; and
combining said plurality of measurement vectors, such that a resultant vector is created/mathematically determined;
wherein said resultant vector represents a proptosis measurement.

2. The method of claim 1, further comprising:
by an ophthalmic measurement device:
determining, based on said proptosis measurement, one of a mid-proptosis, a mild proptosis, and a severe proptosis.

3. The method of claim 1, wherein said digital image providing comprises capturing said digital image with a digital camera.

4. The method of claim 1, wherein said digital image providing comprises:
providing a photograph;
scanning said photograph, such that said digital image is created.

5. The method of claim 1, wherein said one or more distinguishing auricle features comprise one or more of a lobule, a helix, and a tragus.

6. The method of claim 1, wherein said one or more distinguishing oculus features comprise one or more of a pupil, a canal of Schlemm, an iris, and a cornea.

7. The method of claim 1, wherein said plurality of anatomical auricle points comprise: a triangular fossa; a superior crus of antihelix; a helix; a scapha; an antihelix stem; a cymba, a cavum, an antitragus; a lobe; an incisura; a tragus; a crus helix; and an inferior crus of antihelix.

8. The method of claim 1, wherein said plurality of measurement vectors is a minimum of three measurement vectors.

9. A non-transitory computer readable medium with computer executable instructions for ophthalmic measuring, the computer readable medium having computer executable instructions comprising:
providing an electronic application;
providing a digital image;
receiving, by said electronic application, said digital image;
wherein said digital image comprises a depiction of an auricle and an oculus;
wherein said auricle and said oculus are positioned on a same side of a face;
converting, by said electronic application, said digital image, such that said depiction of said auricle and said oculus are rendered into a digital representation of said auricle and into a digital representation of said oculus;
processing, by said electronic application, said digital representation of said auricle, such that said electronic application identifies a plurality of anatomical auricle reference points;
processing, by said electronic application, said digital representation of said oculus, such that said electronic application identifies a corneal apex of said digital representation of said oculus;
creating, by said electronic application, a plurality of measurement vectors, each of which originates from one of said plurality of anatomical auricle reference points and terminates at said corneal apex;
combining, by said electronic application, said plurality of measurement vectors, such that a resultant vector, is created/mathematically determined; and
wherein said resultant vector represents a proptosis measurement.

10. The instructions of claim 9, further comprising:
determining, by said electronic application, a diagnosis of one of a mid-proptosis, a mild proptosis, and a severe proptosis, based on said resultant vector.

11. The instructions of claim 9, wherein said digital image providing comprises capturing said digital image with a digital camera.

12. The instructions of claim 9, wherein said digital image providing comprises:
providing a photograph;
scanning said photograph, such that said digital image is created.

13. The instructions of claim 9, wherein said processing, by said electronic application to identify said plurality of anatomical auricle reference points comprises:
defining, by said electronic application, a plurality of boundaries of said digital representation of said auricle by identifying, by said electronic application, one or more distinguishing auricle features to enable recognition of said plurality of anatomical auricle reference points; and
mapping, by said electronic application, said digital representation of said auricle by identifying said plurality of anatomical auricle reference points that were recognized.

14. A system for obtaining ophthalmic measurements for the purpose of diagnosing proptosis, comprising:
an ophthalmic measurement device, which comprises a digital camera, a display, an electronic data processing unit, and a memory;
an electronic application, at least a portion of which is stored in said memory and processed by said electronic data processing unit;
wherein said digital camera captures a digital image comprises a depiction of an auricle and of an oculus that are on a same side of a face;
wherein said electronic application, running on said ophthalmic measurement device:
renders, respectively, said depiction of said auricle and said oculus into a digital representation of said auricle and into a digital representation of said oculus;
defines a plurality of boundaries of said digital representation of said oculus by identifying one or more distinguishing oculus features to enable recognition of a corneal apex of said oculus;
defines a plurality of boundaries of said digital representation of said auricle by identifying one or more distinguishing auricle features to enable recognition of a plurality of anatomical auricle reference points;
digitally maps said digital representation of said auricle by identifying said plurality of anatomical auricle reference points that were defined;
creates a plurality of a measurement vectors, wherein each of said plurality of measurement vectors originates from one or more of said plurality of anatomical auricle reference points and extends to said corneal apex; and
combines said plurality of measurement vectors, such that a resultant vector is created;
wherein said resultant vector represents a proptosis measurement.

15. The system of claim 14, further comprising an artificial intelligence engine.

16. The system of claim 14, wherein said electronic application running on said ophthalmic measurement device determines, based on said proptosis measurement, one of a mid-proptosis, a mild proptosis, and a severe proptosis.

17. The system of claim 14, wherein said one or more distinguishing auricle features comprise one or more of a lobule, a helix, and a tragus.

18. The system of claim 14, wherein said one or more distinguishing oculus features comprise one or more of a pupil, a canal of Schlemm, an iris, and a cornea.

19. The system of claim 14, wherein said plurality of anatomical auricle points comprise: a triangular fossa; a superior crus of antihelix; a helix; a scapha; an antihelix stem; a cymba, a cavum, an antitragus; a lobe; an incisura; a tragus; a crus helix; and an inferior crus of antihelix.

20. The system of claim 14, wherein said plurality of measurement vectors is a minimum of three measurement vectors.
